(19) United States
(12) Patent Application Publication
(10) Pub. No.: US 2013/0296723 A1
(43) Pub. Date: Nov. 7, 2013

(54) PORTABLE BLOOD PRESSURE MEASURING APPARATUS AND BLOOD PRESSURE MEASURING METHOD IN PORTABLE TERMINAL

(71) Applicant: SAMSUNG ELECTRONICS CO., LTD., Gyeonggi-do (KR)

(72) Inventors: Jae-Geol CHO, Gyeonggi-do (KR); Se-Dong Min, Seoul (KR); Jae-Pil Kim, Gyeonggi-do (KR); Min-Hyoung Lee, Gyeonggi-do (KR)

(73) Assignee: Samsung Electronics Co., Ltd., Gyeonggi-do (KR)

(21) Appl. No.: 13/827,298

(22) Filed: Mar. 14, 2013

(30) Foreign Application Priority Data
May 3, 2012 (KR) 10-2012-0046844

(51) Int. Cl. A61B 5/021

(52) U.S. Cl. CPC A61B 5/02108
USPC 600/501

(57) ABSTRACT

A method and apparatus for readily measuring a blood pressure without using a cuff includes measuring, by a portable blood pressure measuring apparatus, an electrocardiogram signal and a pulse wave signal, transmitting the measured electrocardiogram signal and pulse wave signal to a portable terminal, calculating, by the portable terminal, a Pulse Transit Time (PTT) and a Pulse Wave Velocity (PWV) using the transmitted electrocardiogram signal and the pulse wave signal, and calculating a blood pressure value based on the PTT and the PWV. Therefore, users may readily measure a blood pressure at any time and place and may be provided with a customized blood pressure measurement result.

### PRIORITY

This application claims priority under 35 U.S.C. §119(a) to Korean Application Serial No. 10-2012-0046844, which was filed in the Korean Intellectual Property Office on May 3, 2012, the contents of which are incorporated herein by reference.

### BACKGROUND OF THE INVENTION

1. Field of the Invention

The present invention relates generally to a blood pressure measuring method and apparatus.

2. Description of the Related Art

Ubiquitous environments that freely connect a network regardless of a time or a location, are currently being provided and are allowing various information to be available to many users for sharing.

In the ubiquitous environments, a health care field (U-healthcare) refers to an environment where a user is provided with a medical service through a network, without visiting a hospital, and a health condition of the user is frequently checked. Therefore, research on the health care field has been actively conducted and various medical devices have been developed.

In addition, as health consciousness has recently increased, users have increasingly measured their health conditions using various medical devices. For example, a user may measure a bio-signal such as blood pressure, heart rate, and pulse.

To prevent hypertension and the like, it is most important to continuously measure blood pressure. To do so, a method has been developed that measures a blood pressure based on oscillation that is generated when a user winds a cuff around an arm of the user and pressurizes or depressurizes an artery using the cuff A device for conveniently measuring blood pressure from a wrist or a finger using a cuff has also been actively studied and commercialized.

An upper arm-type blood pressure measuring apparatus that winds a cuff around an arm of the user is large, and is therefore difficult to carry. Also, a scheme that winds a cuff around a wrist or a finger for easy portability is inconvenient during measurement, due to a line that connects the cuff and a blood pressure measuring apparatus. In particular, blood pressure may sensitively vary based on various conditions such as a cuff characteristic and a user's posture during measurement, which can compromise the accuracy of the measurement.

### SUMMARY OF THE INVENTION

Accordingly, an aspect of the present invention is to solve at least the above-described problems occurring in the prior art, and to provide at least the advantages described below.

An aspect of the present invention is to provide a portable blood pressure measuring method and apparatus for measuring a blood pressure without using a cuff

Another aspect of the present invention is to provide a portable blood pressure measuring method and apparatus that considers a posture of a user during measurement.

Another aspect of the present invention is to provide a portable blood pressure measuring method and apparatus for providing a customized blood pressure monitoring result screen.

In accordance with the present invention, a portable blood pressure measuring apparatus includes an electrocardiogram electrode unit including a first electrode, a second electrode, and a third electrode included in a surface of a body, to measure an electrocardiogram signal through the first electrode through the third electrode, a first sensor unit, included in the same location as the third electrode unit, to measure a pulse wave signal, a second sensor unit to sense a movement of the portable blood pressure measuring apparatus, a controller to determine whether the movement is sensed by the second sensor unit when the electrocardiogram signal is measured by the electrocardiogram electrode unit and the pulse wave signal is measured by the first sensor unit, and to perform controlling so as to measure the electrocardiogram signal and the pulse wave signal when it is sensed that the movement is less than a threshold value, and a wireless communication unit to transmit the measured electrocardiogram signal and pulse wave signal to a portable terminal that measures a blood pressure value using the measured electrocardiogram signal and the pulse wave signal.

In accordance with another aspect of the present invention, a method of measuring a blood pressure in a portable terminal includes receiving an electrocardiogram signal and a pulse wave signal from a portable blood pressure measuring apparatus when a blood pressure measurement application is executed, calculating a Pulse Transit Time (PTT) and a Pulse Wave Velocity (PWV) using the received electrocardiogram signal and the pulse wave signal, calculating a blood pressure value using the calculated PTT and PWV, and outputting the calculated blood pressure value and a blood pressure measurement result.

### BRIEF DESCRIPTION OF THE DRAWINGS

The above and other aspects, features, and advantages of the present invention will be more apparent from the following detailed description taken in conjunction with the accompanying drawings, in which:

FIG. 1 illustrates a configuration of a portable blood pressure measuring apparatus and a portable terminal according to embodiments of the present invention;

FIG. 2 is a perspective view of the portable blood pressure measuring apparatus of FIG. 1;

FIG. 3 illustrates an example of a method of gripping the portable blood pressure measuring apparatus of FIG. 2;

FIG. 4 illustrates a method of measuring a PTT using an electrocardiogram signal and a pulse wave signal;

FIG. 5 illustrates operations of a portable blood pressure measuring apparatus according to a first embodiment of the present invention;

FIG. 6 illustrates operations of a portable terminal according to the first embodiment of the present invention;

FIG. 7 illustrates operations of a portable blood pressure measuring apparatus according to a second embodiment of the present invention; and

FIG. 8 illustrates an example of a blood pressure measurement screen of a portable terminal according to embodiments of the present invention.

### DETAILED DESCRIPTION OF EMBODIMENTS OF THE PRESENT INVENTION

Hereinafter, embodiments of the present invention will be described with reference to the accompanying drawings. In the following description, the same elements will be designated by the same reference numerals although they are shown in different drawings. Various specific definitions found in the following description are provided only to assist the general understanding of the present invention, and it is apparent to those skilled in the art that the present invention can be implemented without such definitions. In the following description of the present invention, a detailed description of known functions and configurations incorporated herein will be omitted for the sake of clarity and conciseness.

The present invention provides a method and apparatus for readily measuring blood pressure without a cuff, by measuring an electrocardiogram signal and a pulse wave signal through a portable blood pressure measuring apparatus, transmitting the measured electrocardiogram signal and the pulse wave signal to a portable terminal, calculating a PTT and a PWV in the portable terminal based on the transmitted information, and calculating a blood pressure value based on the PTT and the PWV. Therefore, a user may readily measure a blood pressure at any time and place, and may be provided with a customized blood pressure measurement result.

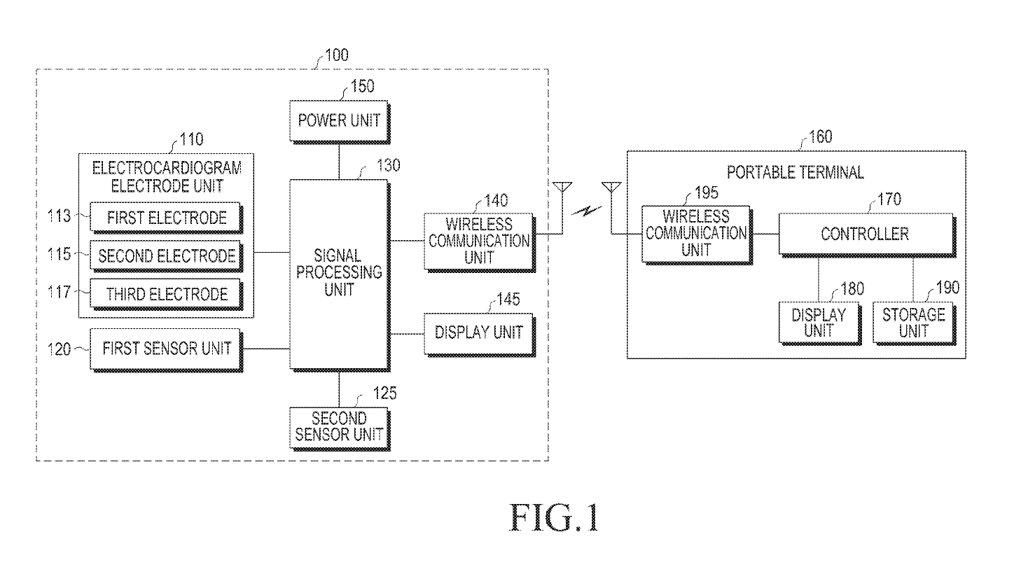

FIG. 1 describes component elements and operations of a portable blood pressure measuring apparatus and a portable terminal that wirelessly communicates with the portable blood pressure measuring apparatus. The portable blood pressure measuring apparatus refers to a portable medical device, that is, a card-type cuffless measuring apparatus. The portable blood pressure measuring apparatus may be contained in a portable phone, an MPEG-Layer Audio 3 (MP3) player, or a portable terminal case, and may be an accessory attached to the portable terminal.

Examples of the portable terminal include a portable phone, a smart phone, a tablet Personal Computer (PC), a PC, a notebook, a digital sound source playback apparatus, a Portable Multimedia Player (PMP), and any device that is capable of transceiving data through communication with the portable blood pressure measuring apparatus.

Referring to FIG. 1, a portable blood pressure measuring system includes a portable blood pressure measuring apparatus 100 and a portable terminal 160.

The portable blood pressure measuring apparatus 100 includes an electrocardiogram electrode unit 110, a first sensor unit 120, a second sensor unit 125, a signal processing unit 130, a wireless communication unit 140, a display unit 145, and a power unit 150. The signal processing unit 130 may also be referred to as a controller.

The electrocardiogram electrode unit 110 is configured of a first electrode 113 and a second electrode 115, which have different polarities from each other, and may be in contact with a portion of a body of a user so as to measure an electrocardiogram signal from among bio-signals. The electrocardiogram electrode unit 110 further includes a third electrode 117, and may reduce an error of an electrocardiogram signal measured through the first electrode 113 and the second electrode 115.

It is assumed that the first electrode 113 has a positive(+) polarity (or a positive potential), the second electrode 115 has a negative(−) polarity (or a negative potential), and the third electrode 117 has a ground polarity (or a ground potential). The electrocardiogram electrode unit 110 may measure an electrocardiogram signal associated with a change, over time, in an action potential of a cardiac muscle cell generated based on a heartbeat, using a potential difference of the first electrode 113 and the second electrode 115 with respect to the third electrode 117.

The electrocardiogram electrode unit 110 is mounted on the card-type portable blood pressure measuring apparatus 100 and is in direct contact with a portion of a body of the user, in which case the electrocardiogram electrode unit 110 measures an electrocardiogram signal and transmits the measured signal to the signal processing unit 130.

Although it is described that the electrocardiogram electrode unit 110 measures an electrocardiogram signal when the electrocardiogram electrode unit 110 senses contact with a portion of the body of the user, and transmits the measured signal to the signal processing unit 130, the electrocardiogram electrode unit 110 may measure an electrocardiogram signal when a start button is pressed by the user, and then transmit the measured signal to the signal processing unit 130.

The first sensor unit 120 is formed of a photo sensor, such as a reflection-type photo sensor. The first sensor unit 120 is in contact with a portion of the body of the user, such as a finger, and measures a pulse wave signal from among bio-signals and transmits the measured signal to the signal processing unit 130.

The electrocardiogram electrode unit 110 and the first sensor unit 120 may be mounted on an external side of the portable blood pressure measuring apparatus 100 so as to be in direct contact with a portion of the body of the user.

The second sensor unit 125 is for sensing a motion of the portable blood pressure measuring apparatus 100, and may be embodied as an inertial sensor such as an accelerometer, a gyroscope, a shock sensor, a tilt sensor, an altimeter, a gravity sensor, a terrestrial magnetism, a combination thereof, or as another type of a sensor that is capable of sensing a movement or a tilt of the portable blood pressure measuring apparatus 100.

The second sensor unit 125 distinguishes a movement of the portable blood pressure measuring apparatus 100, such as a tilting motion, based on a sensor signal collected by an acceleration sensor or a gyro-sensor, for example, and transmits a corresponding motion signal to the signal processing unit 130.

The signal processing unit 130 is included in the portable blood pressure measuring apparatus 100, and controls general operations and conditions of component elements of the portable blood pressure measuring apparatus 100. Particularly, when power is supplied through the power unit 150, the signal processing unit 130 determines whether a contact is sensed in the electrocardiogram electrode unit 110 and the first sensor unit 120, or determines whether the start button is pressed. When a contact or a press on the start button is sensed, the signal processing unit 130 performs primary signal processing on an electrocardiogram signal and a pulse wave signal corresponding to bio-signals received from the electrocardiogram electrode unit 110 and the first sensor unit 120 for communication with the portable terminal 160, and transmits the signal to the portable terminal 160 through the wireless communication unit 140.

In this example, when a contact or a press on the start button is sensed, the signal processing unit 130 calculates a movement value associated with a degree of a tilt or a distance of a movement based on a signal received from the second sensor unit 125. When the calculated movement value is greater than or equal to a threshold value, measurement of the electrocardiogram signal and the pulse wave signal is controlled to be stopped and the signal processing unit 130 disregards an electrocardiogram signal and a pulse wave signal even though the electrocardiogram signal is received from the electrocardiogram electrode unit 110 and the pulse wave signal is received from the first sensor unit 120. That is, the signal processing unit 130 does not perform signal processing on the electrocardiogram signal and the pulse wave signal. A calculated movement value being greater than or equal to a threshold value corresponds to when a posture of the user during the measurement is tilted or when the user is in motion.

When the movement of the user is sensed, the signal processing unit 130 performs controlling to output a notification message on the display unit 145 or to output an alert sound so that the user may measure a blood pressure in a stable posture. The notification message or the alert sound performs a function of guiding or inducing the user to change a posture for the measurement.

The wireless communication unit 140 performs a communication function between the portable blood pressure measuring apparatus 100 and the portable terminal 160. The wireless communication unit 140 may perform a short distance communication such as Bluetooth®, Zigbee®, and Near Field Communication (NFC) and may perform a wireless communication function such as Wireless Local Area Network (WLAN) and Wifi. The wireless communication unit 140 transmits a measured value to the portable terminal 160 of the user, but may transmit the measured value to a portable terminal of a medical expert or a medical institution. When the measured value is transmitted to the portable terminal of the medical expert or the medical institution, the health condition of the user may be monitored and a result of the monitoring may be fed back to the user.

The display unit 145 displays various information associated with operations of the portable blood pressure measuring apparatus 100. For example, the display unit 145 displays information associated with measuring an electrocardiogram signal and a pulse wave signal. In this example, the portable blood pressure measuring apparatus 100 is light-weight and small, making it easily portable. Thus, a size of the display unit 145 is relatively small. Accordingly, various screens for information associated with a measurement result obtained in the portable blood pressure measuring apparatus 100 may be output through a display unit 180 of the portable terminal 160.

When the signal processing unit 130 of the portable blood pressure measuring apparatus 100 supports a function of calculating a blood pressure value using an electrocardiogram signal and a pulse wave signal, the display unit 145 may be embodied to also display the electrocardiogram signal and the pulse wave signal as a graph, and may output a screen displaying a blood pressure measurement result.

The portable terminal 160 includes a controller 170, the display unit 180, a storage unit 190, and a wireless communication unit 195.

The controller 170 calculates a blood pressure value by calculating a PTT and a PWV based on an electrocardiogram signal and a pulse wave signal received from the portable blood pressure measuring apparatus 100.

The display unit 180 displays various information associated with conditions and operations of the portable terminal 160 and the portable blood pressure measuring apparatus 100, based on controlling of the controller 170. The display unit 180 according to embodiments of the present invention displays the blood pressure value calculated based on the electrocardiogram signal and the pulse wave signal, and user's health condition information based on the calculated blood pressure value. The display unit 180 displays, to the user, a screen that provides an exercise prescription based on the health condition and an analysis result obtained by monitoring a blood pressure value before/after the exercise.

The storage unit 190 stores an operating system of the portable terminal 160, various applications, information input to the portable terminal 160 and internally generated information. For example, the storage unit 190 stores continuously calculated blood pressure values and health information associated with the blood pressure value, and both of these parameters may be used for continuously checking the health condition of the user.

The storage unit 190 generally includes a program area (not shown) and a data area (not shown). The program area includes programs for calculating a blood pressure value, configuring a change in a health condition of the user in a screen based on the calculated blood pressure value, and providing an exercise prescription and an analysis result by monitoring and analyzing a blood pressure value before and after an exercise when the prescribed exercise is executed. In the present invention, the programs are referred to as a blood pressure measurement application.

The data area stores data generated as the portable terminal 160 is used, and may store data generated when a blood pressure measurement application is executed according to embodiments of the present invention, for example, an electrocardiogram signal, a pulse wave signal, a PTT, a PWV, or a blood pressure value.

The wireless communication unit 195 wirelessly transmits data from the controller 170, or wirelessly receives data from the portable blood pressure measuring apparatus 100 so as to transfer the data to the controller 170.

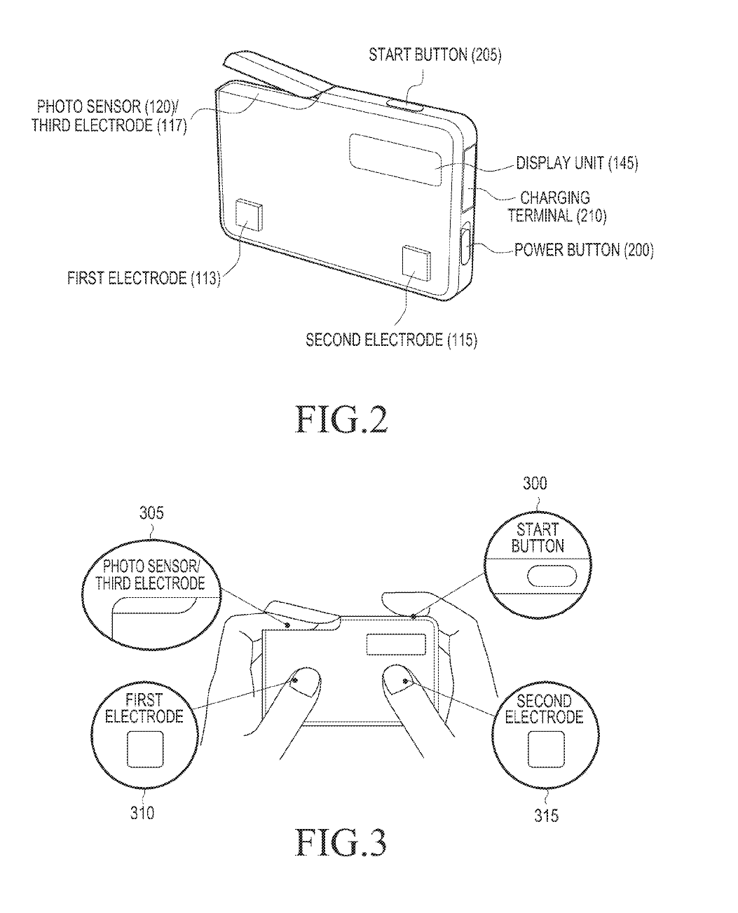

FIG. 2 is a perspective view of the portable blood pressure measuring apparatus 100 of FIG. 1. As illustrated in FIG. 2, the portable blood pressure measuring apparatus 100 includes two electrocardiogram electrodes 113 and 115 having negative and positive poles in a surface of a body, for example, a foreside, and includes the photo sensor 120 and the third electrode 117 in the body, for example, an upper portion, so that an electrocardiogram signal and a pulse wave signal are readily measured. The portable blood pressure measuring apparatus 100 further includes a power button 200, a start button 205, and a charging terminal 210.

When the user desires to measure a blood pressure, the user brings a finger into contact with the photo sensor 120, brings another finger from the same hand into contact with the first electrode 113, and brings a finger of another hand into contact with the second electrode 115. When the contacts are maintained, when the user presses the start button 205, an electrocardiogram signal and a pulse wave signal are measured through the first electrode 113 through the third electrode 117, and the photo sensor 120.

FIG. 2 illustrates an example in which two electrocardiogram electrodes are disposed on the front of the body of the apparatus, and a single electrocardiogram electrode and a photo sensor are disposed on the upper portion of the body of the apparatus. The location where the electrocardiogram electrodes are disposed may be changed based on a gripping method (or grip posture) of the user who grips the portable blood pressure measuring apparatus 100. That is, the electrocardiogram electrodes are preferably disposed on locations with which the user is capable of bringing three fingers into contact.

FIG. 3 illustrates a method of gripping the portable blood pressure measuring apparatus 100 where the electrocardiogram electrodes are disposed as illustrated in FIG. 2. As illustrated in FIG. 3, a photo sensor and a third electrode are disposed on an area 305 of an upper portion of a body of the portable blood pressure measuring apparatus 100, with which a left index finger of the user is in contact, and a first electrode and a second electrode are disposed on areas 310 and 315 of the front of the body, with which thumbs of both hands are in contact. In this state, measurement is started when the user presses a start button disposed on an area 300 with which a right thumb is in contact.

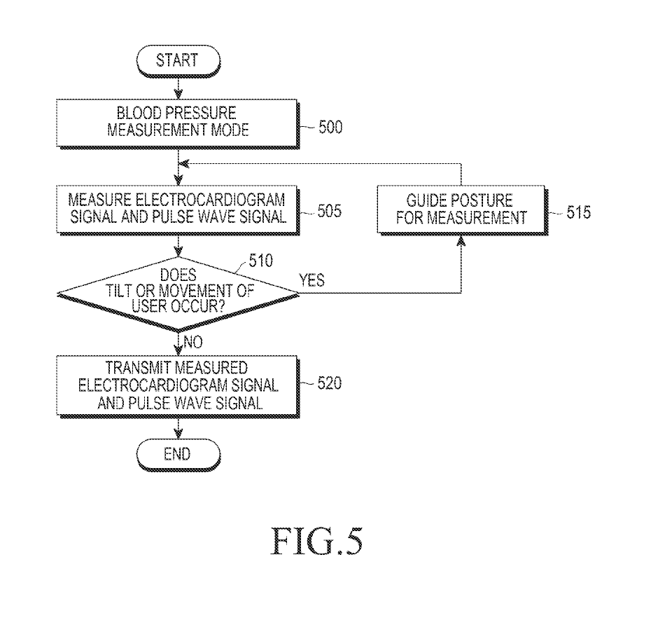

FIG. 5 illustrates operations of a portable blood pressure measuring apparatus according to a first embodiment of the present invention. Referring to FIG. 5, the portable blood pressure measuring apparatus 100 proceeds with a blood pressure measuring mode in step 500, and measures an electrocardiogram signal and a pulse wave signal in step 505 when a press on a start button is sensed. During measuring, it is determined in step 510 whether the user is in motion or the portable blood pressure measuring apparatus 100 is tilted. For this, the signal processing unit 130 of the portable blood pressure measuring apparatus 100 calculates a movement value associated with a degree of the tilt or a distance of the movement based on the signal received through the second sensor unit 125. In this example, a threshold value may be determined in advance, for determining whether the portable blood pressure measuring apparatus 100 is tilted or the user is in motion. The threshold value may be adjusted by improving accuracy of the portable blood pressure measuring apparatus 100.

When the calculated movement value is greater than or equal to the threshold value, for example, when the degree of the tilt is greater than or equal to an angle or the user is in motion, a posture for measurement is indicated in step 515 so that the user may measure a blood pressure in a stable posture. When the portable blood pressure measuring apparatus 100 is tilted or the user is in motion, it is difficult to expect an accurate measured blood pressure value and thus, a measured electrocardiogram signal and a pulse wave signal may not be transmitted to the portable terminal 160. Unlike the above, when it is determined that a movement of the portable blood pressure measuring apparatus 100 does not exist, while an electrocardiogram signal and a pulse wave signal are measured, the portable blood pressure measuring apparatus 100 transmits the measured electrocardiogram signal and pulse wave signal to the portable terminal 160 in step 520.

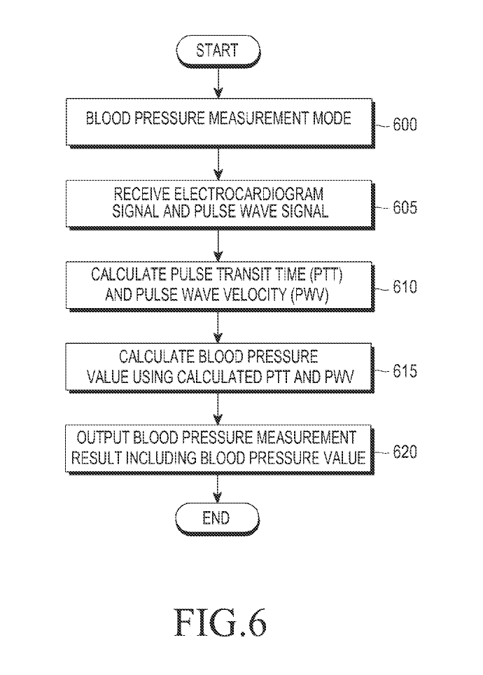

FIG. 6 illustrates operations of a portable terminal according to the first embodiment of the present invention.

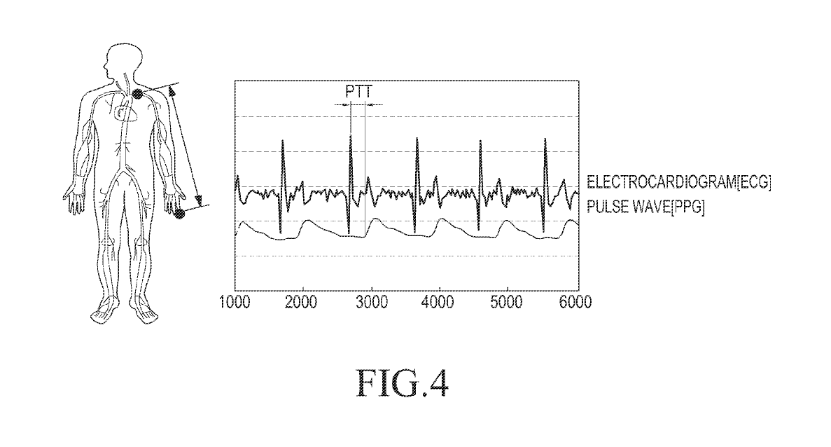

Referring to FIG. 6, the portable terminal 160 proceeds with a blood pressure measuring mode in step 600 as a blood pressure measurement application is executed, and receives an electrocardiogram signal and a pulse wave signal from the portable blood pressure measuring apparatus 100 in step 605. The portable terminal 160 calculates a PTT and a PWV using the received electrocardiogram signal and pulse wave signal in step 610. The remaining steps of FIG. 6 will be explained after FIG. 4 is discussed.

FIG. 4 illustrates a method of measuring a PTT using an electrocardiogram signal and a pulse wave signal. As illustrated in FIG. 4, a difference in time between an R peak of an electrocardiogram and a start point of a pulse wave signal measured by a finger that is in contact with a photo sensor included in the portable blood pressure measuring apparatus 100 is referred to as a PTT. The PTT is a time expended when blood leaves the heart and arrives at a peripheral part such as a finger, that is, a time expended when a pulse wave signal is transferred to a point of the measurement from contraction of a ventricle, and is calculated based on an electrocardiogram signal and a pulse wave signal.

The PTT may be calculated by calculating a difference in time between a peak of an electrocardiogram signal and a start point of a pulse wave signal.

A PWV may be calculated by dividing, by the PTT, a distance from the heart to a point where a pulse wave signal is measured, that is, a length of a blood vessel.

The PWV may be expressed based on Equation (1), as follows.

$$PWV = \sqrt{\frac{Eh}{2 ρ R}} \qquad ( 1 )$$

In Equation (1), R denotes a blood vessel radius, h denotes a wall thickness, and ρ denotes a blood density. Also, E denotes a modulus of Elasticity, Young's modulus, of an artery associated with a pressure.

The PWV may be expressed by a function of E. The PWV may vary based on a blood pressure, a blood vessel diameter, and a blood vessel thickness, such as when the PWV becomes higher as a blood vessel is thicker. In general, as the elasticity of a blood vessel is lower, the PWV becomes higher. In particular, a PWV of a main artery is an index for early detection of such ailments as blood vessel aging, arteriosclerosis, hypertension, diabetes, hyperlipidemia, and a kidney ailment.

E may be expressed by Equation (2), as follows:

E=Eₒ exp(αP) (2)

In Equation (2), E0 and a denote a constant, and P denotes an internal pressure of a blood vessel. Therefore, the PTT may be expressed by Equation (3) by substituting Equation (2) in Equation (1).

$$PTT = \frac{L}{PWV} = L \sqrt{\frac{2 ρ R}{{E}_{o} h}} exp ( - \frac{α}{2} P ) \qquad ( 3 )$$

In Equation (3), L denotes a length of a blood vessel, P denotes an internal pressure of a blood vessel, and E0 denotes an Young's modulus when a cuff pressure is 0. α denotes a constant corresponding to a modulus of elasticity of a blood vessel, and the internal pressure P of a blood vessel indicates the same value as an average blood pressure since a cuff is not used in embodiments of the present invention. Also, L may be calculated by actual measuring, or based on a regression Equation using gender and height. An example of the regression Equation is Equation (4), and the regression Equation used in the present invention is not limited thereto.

L=0.4861×Height+0.6337 (cm) (4)

In Equation (4), Height denotes a user's height. Equation (4) illustrates a regression Equation indicating a length from the heart of a Korean male to his finger.

Therefore, when a PTT is calculated based on an electrocardiogram signal and a pulse wave signal, Equation (4) is substituted in Equation (3). The length of the blood vessel may be calculated by Equation (4) and the PTT is calculated based on an electrocardiogram and a pulse wave signal and thus, a PWV may also be calculated by substituting the calculated length of the blood vessel and the PTT in Equation (3).

Referring back to FIG. 6, when the PTT and the PWV are calculated, the portable terminal 160 calculates a blood pressure value based on the calculated PTT and PWV in step 615. In particular, the blood pressure value is calculated by substituting the PTT and the PWV in Equation (3). The blood pressure value denotes the average blood pressure P in Equation (3).

Therefore, when the blood pressure value is calculated, the portable terminal 160 outputs a blood pressure measurement result including the blood pressure value in step 620. For example, when the user continuously measures an electrocardiogram signal and a pulse wave signal, a change in a PTT and a PWV may be recognized. Accordingly, when the continuous measuring shows that the PWV increases when compared to a previous average, it is recognized that E associated with the modulus of elasticity α of a blood vessel of the user is changed through Equation (1). For example, a degree of hardening of an artery refers to an extent of a thickness of an inside wall of a blood vessel that becomes thicker and an extent of elasticity that becomes lower as a foreign substance is accumulated in the inside wall of the blood vessel. Thus, the degree of hardening of an artery may be estimated by measuring the elasticity of the blood vessel. A well known constant may be substituted in a modulus of elasticity of a blood vessel.

As another example, a value calculated based on measured data accumulated by continuous measurement learning may be substituted. In a blood pressure measuring apparatus having a cuff, a value calculated using a blood pressure in a cuff section, an initial PTT, a length of the cuff, a length of an artery, or an increase in a PTT in the cuff section may be substituted in a modulus of elasticity of a blood vessel.

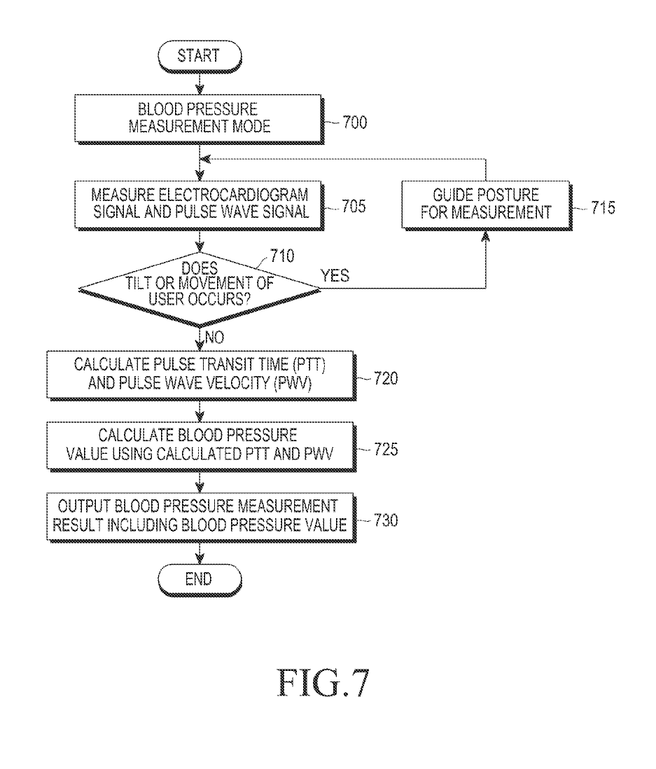

FIG. 7 illustrates operations of a portable blood pressure measuring apparatus according to a second embodiment of the present invention.

Unlike the first embodiment of the present invention, in FIG. 7, the portable blood pressure measuring apparatus 100 calculates a PTT and a PWV as illustrated in steps 720 and 730, calculates a blood pressure value using the PTT and the PWV, and outputs a blood pressure measurement result, as opposed to outputting the result through the portable terminal 160. Therefore, operations in steps 700 through 715 are the same as operations in steps 500 through 515 of FIG. 5 and thus, detailed descriptions thereof will be omitted. As another example, when the portable blood pressure measuring apparatus 100 calculates a blood pressure value and transmits the calculated blood pressure to the portable terminal 160, the portable terminal 160 may output a blood pressure measurement result.

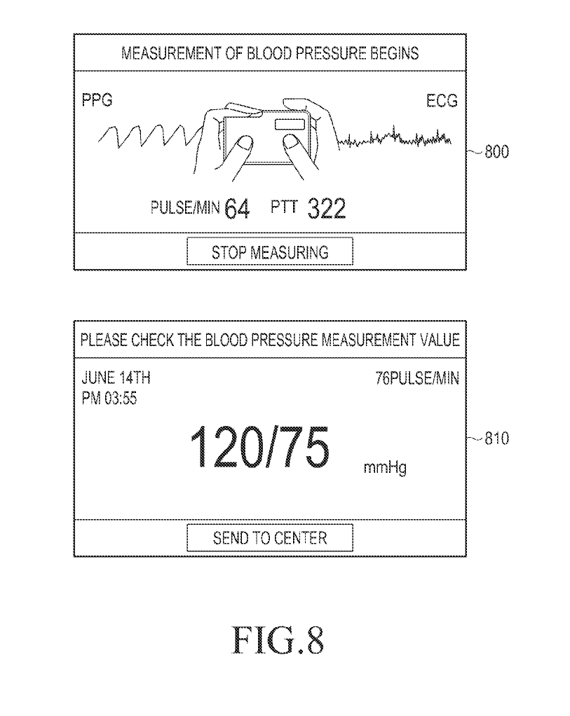

FIG. 8 illustrates an example of a blood pressure measurement screen of a portable terminal. A blood pressure measurement screen 800 illustrates when an electrocardiogram signal and a pulse wave signal are measured after a mode is changed into a blood measurement mode. A blood pressure measurement screen 810 illustrates a blood pressure measurement result. The portable terminal 160 according to embodiments of the present invention stores continuously calculated blood pressure values in the storage unit 190, records a point in time of measuring blood pressure by determining whether the point in time of measuring the blood pressure of a user corresponds to before or after performing exercise, based on health information associated with the blood pressure value and the like, and outputs a screen that shows a health condition of the user through records.

As described in the foregoing, according to embodiments of the present invention, an appropriate action against a dangerous change in health of the user may be taken in advance by frequently checking a blood pressure of the user through the portable blood pressure measuring apparatus 100 and outputting a result corresponding to a change in a blood pressure value through the portable terminal 160. Thus, health of the user is stably monitored.

In addition, according to embodiments of the present invention, a blood pressure is readily measured through the portable blood pressure measuring apparatus, and the user may readily recognize a health condition of the user in addition to various information that may be provided by the portable blood pressure measuring apparatus through use of a display unit of the portable terminal 160 which displays manipulated contents, since the display unit of the portable terminal 160 improves visibility through a large and detailed picture.

According to embodiments of the present invention, blood pressure is conveniently measured within a short period of time without using a cuff, and users may readily measure a blood pressure at any time and place since the portable blood pressure measuring apparatus is portable.

According to embodiments of the present invention, a sensor is installed in the portable blood pressure measuring apparatus so as to inform the user of an unstable posture or movement of the user when the unstable posture or movement of the user occurs during the measurement. Thus, the user may be guided to measure a blood pressure in a stable posture.

According to embodiments of the present invention, information associated with an electrocardiogram and a pulse wave measured through the portable blood pressure measuring apparatus in which a sensor is installed is transmitted to a portable terminal, which determines whether a blood pressure value calculated based on the transmitted information corresponds to before performing exercise or after performing exercise, and may support a function of monitoring a blood pressure improved through continuous exercise.

The above-described embodiments of the invention may be embodied as hardware, software or a combination of hardware and software. Software may be stored in a volatile or non-volatile storage device such as Read Only Memory (ROM) and the like irrespective of erasing or rewriting, a memory such as a Random Access Memory (RAM), a memory chip, a device, and a integrated circuit, or a storage medium that is capable of performing optical or magnetic recording and machine-reading such as Compact Disc (CD), Digital Versatile Disc (DVD), optical disc, and magnetic tape. A storage unit that may be included in a portable terminal may be an example of machine-readable storage media that are suitable for storing a program including instructions to implement the embodiments, or programs. Therefore, the invention may include a program including a code to implement an apparatus or a method claimed in a claim of the specification, and a machine-readable storage medium including the program, for example, a computer-readable storage medium. The program may be transferred electronically through a medium such as a communication signal transferred through a wired or wireless connection, and the invention may appropriately include an equivalent medium.

A portable blood pressure measuring apparatus or a portable terminal herein may receive the program from a program providing device that is a wired or wirelessly connected, and may store the program. The program providing device may include a program including instructions to instruct the portable blood pressure measuring apparatus or the portable terminal to perform a blood pressure measuring method, a memory storing information required for the blood pressure measuring method and the like, a communication unit to perform wired or wireless communication with the portable blood pressure measuring apparatus or the portable terminal, and a controller to transmit the program to the portable blood pressure measuring apparatus and the portable terminal, automatically or in response to the request from the portable blood pressure measuring apparatus or the portable terminal.

While the present invention has been shown and described with reference to certain embodiments thereof, it will be understood by those skilled in the art that various changes in form and details is made therein without departing from the spirit and scope of the present invention as defined by the appended claims. Therefore, several modifications are possible without departing from the gist of the present invention as defined by the appended claims. It should be understood that the modifications remain within the technical ideas and overviews of the invention.

What is claimed is:

1. A portable blood pressure measuring apparatus, the apparatus comprising:
an electrocardiogram electrode unit, including a first electrode, a second electrode, and a third electrode included in a surface of a body, which measure an electrocardiogram signal through the first electrode through the third electrode;
a first sensor unit which measures a pulse wave signal;
a second sensor unit which senses a movement of the portable blood pressure measuring apparatus;
a controller which determines whether the movement is sensed by the second sensor unit during measuring the electrocardiogram signal and the pulse wave signal, and which performs controlling so as to measure the electrocardiogram signal and the pulse wave signal when it is sensed that the movement is less than a threshold value; and
a wireless communication unit which transmits the measured electrocardiogram signal and pulse wave signal to a portable terminal that measures a blood pressure value using the measure electrocardiogram signal and the pulse wave signal.

2. The apparatus of claim 1, wherein the controller stops measuring the electrocardiogram signal and the pulse wave signal when the movement is greater than or equal to the threshold value.

3. The apparatus of claim 1, wherein the controller performs controlling to output a notification associated with a posture during measuring when the movement is greater than or equal to the threshold value.

4. The apparatus of claim 1, wherein the first sensor unit corresponds to a photo sensor.

5. The apparatus of claim 1, wherein the first electrode, the second electrode, and the third electrode have a positive pole, a negative pole, and a ground pole, respectively.

6. The apparatus of claim 1, wherein the portable terminal calculates a pulse transit time and a pulse wave velocity using the electrocardiogram signal and the pulse wave signal received from the portable blood pressure measuring apparatus, and calculates a blood pressure value based on the calculated pulse transit time and pulse wave velocity.

7. The apparatus of claim 6, wherein the portable terminal outputs the calculated blood pressure value and a blood pressure measurement result.

8. The apparatus of claim 6, wherein the pulse transit time is calculated by computing a difference in time between a peak of the electrocardiogram signal and a start point of the pulse wave signal.

9. The apparatus of claim 6, wherein the pulse wave velocity is calculated by dividing, by the pulse transit time, a length of a blood vessel corresponding to a distance from a heart of a user to a point where the pulse wave signal is measured.

10. The apparatus of claim 9, wherein the length of the blood vessel is calculated by measuring the distance from the heart of the user to the point where the pulse wave signal is measured, or based on a regression equation using a gender and a height of the user.

11. A method of measuring a blood pressure in a portable terminal, the method comprising:
receiving an electrocardiogram signal and a pulse wave signal from a portable blood pressure measuring apparatus when a blood pressure measurement application is executed;
calculating a pulse transit time and a pulse wave velocity using the received electrocardiogram signal and the pulse wave signal;
calculating a blood pressure value using the calculated pulse transit time and pulse wave velocity; and
outputting the calculated blood pressure value and a blood pressure measurement result.

12. The method of claim 11, wherein the pulse transit time is calculated by computing a difference in time between a peak of the electrocardiogram signal and a start point of the pulse wave signal.

13. The method of claim 11, wherein the pulse wave velocity is calculated by dividing, by the pulse transit time, a length of a blood vessel corresponding to a distance from a heart of a user to a point where the pulse wave signal is measured.

14. The method of claim 13, wherein the length of the blood vessel is calculated by measuring the distance from the heart of the user to the point where the pulse wave signal is measured, or based on a regression equation using a gender and a height of the user.

15. The method of claim 11, wherein receiving the electrocardiogram signal and the pulse wave signal from the portable blood pressure measuring apparatus comprises:
measuring the electrocardiogram signal through an electrocardiogram electrode unit including a first electrode, a second electrode, and a third electrode included in a surface of a body of the portable blood pressure measuring apparatus;
measuring the pulse wave signal through a first sensor unit; and
receiving the measured electrocardiogram signal and pulse wave signal.

16. The method of claim 15, further comprising:
sensing a movement of the portable blood pressure measuring apparatus during measuring the electrocardiogram signal and the pulse wave signal;
and measuring the electrocardiogram signal and the pulse wave signal when it is sensed that the movement is less than a threshold value.

17. The method of claim 16, further comprising:
stopping measuring the electrocardiogram signal and the pulse wave signal when the movement is greater than or equal to a threshold value.
